(19) United States
(12) Patent Application Publication
(10) Pub. No.: US 2026/0224592 A1
(43) Pub. Date: Aug. 6, 2026

(54) COMPOSITIONS AND METHODS FOR TREATING SEVERE HYPERTRIGLYCERIDEMIA

(71) Applicant: SHENZHEN HIGHTIDE BIOPHARMACEUTICAL LTD., Shenzhen (CN)

(72) Inventors: Liping Liu, Shenzhen (CN); Ru Bai, Shenzhen (CN); Leigh MacConell, Shenzhen (CN)

(21) Appl. No.: 19/151,527

(22) PCT Filed: Mar. 1, 2024
(86) PCT No.: PCT/CN2024/079562

§ 371 Date: Jul. 28, 2025

Related U.S. Application Data
(60) Provisional application No. 63/449,249, filed on Mar. 1, 2023.

(51) Int. Cl. A61K 31/575; A61K 31/155; A61K 31/4375; A61K 38/28; A61K 45/06; A61P 1/16; A61P 3/06

(52) U.S. Cl. CPC A61K 31/575; A61K 31/155; A61K 31/4375; A61K 38/28; A61K 45/06; A61P 1/16; A61P 3/06

(57) ABSTRACT

The invention provides pharmaceutical compositions and methods of use of berberine ursodeoxycholate (BUDC) for treating severe hypertriglyceridemia or an associated diseases or disorder, as monotherapy or in combination therapy with other agents or an adjuvant.

### PRIORITY CLAIMS AND RELATED PATENT APPLICATIONS

This application claims the benefit of priority to U.S. Provisional Application Ser. No. 63/449,249, filed Mar. 1, 2023, the entire content of which is incorporated herein by reference.

### TECHNICAL FIELD OF THE INVENTION

The invention generally relates to pharmaceutical compositions and methods for therapeutic uses thereof. In particular, the invention relates to pharmaceutical compositions and methods of use of berberine ursodeoxycholate (BUDC) for treating severe hypertriglyceridemia or an associated diseases or disorder, as monotherapy or in combination therapy with other agents or an adjuvant.

### BACKGROUND OF THE INVENTION

Hypertriglyceridemia is a common condition characterized by high levels of triglycerides in the bloodstream. Patients can fluctuate between hypertriglyceridemic states: given an appropriate metabolic stress, mild or moderate hypertriglyceridemia can deteriorate into severe hypertriglyceridemia. Patients with severe hypertriglyceridemia (SHTG) have triglyceride levels more than three times the normal level. SHTG can lead to multiple serious conditions, including cardiovascular disease and acute pancreatitis, in part due to associated risk factors such as obesity and type 2 diabetes mellitus. Higher triglyceride levels have been linked to increased risk of acute pancreatitis, a potentially life-threatening complication of SHTG. (Stefanutti, et al. 2013 Ther Apher Dial 7(2):130-7; Pejic, et al. 2006 J Am Board of Family Med 19(3):310-316; Yuan, et al. 2007 Canadian MedAssoc'n J 176(8): 1113-1120; Hegele 2001 Am J Hum Genet 2001; 69:1161-77; Hodis, et al. 1999 Clin Cardiol 22:II15-20; American College of Cardiology. Hypertriglyceridemia Management According to the 2018 AHA/ACC Guideline.)

Diabetes mellitus (DM), a.k.a. diabetes, is a group of disorders characterized by high blood sugar (glucose) levels because the body does not produce enough or respond normally to insulin. It has become pandemic with an estimate of over 300 million people worldwide living with diabetes today. Diabetes can cause a wide range of health complications. Among the three main types of diabetes, type 1 diabetes, type 2 diabetes, and gestational diabetes, type 2 diabetes is the most common form of diabetes accounting for 90-95% of cases. Type 2 diabetes (T2DM) is characterized by impaired insulin secretion, increased hepatic glucose production, and decreased response of peripheral tissues to insulin, i.e., insulin resistance. Although treatments are available for the management of type 2 diabetes, they are often accompanied by various side effects. An optimal therapy should be safe and include early initiation of combination drugs with complimentary mechanisms of action. In the U.S., among individuals with SHTG, 15% had T2DM. (Christian, et al. 2011 Am J Cardiol 107(6):891-7; Kharroubi, et al. 2015 World J Diabetes 6(6):850-867; Olokoba, et al. 2012 Oman Med J 27(4):269-273.)

Despite continued efforts and meaningful progress over the past decades in the understanding and management of HTG, patients with SHTG and with both SHTG and diabetes continue to face an increased risk of, and many do suffer from, a number of serious complications.

Thus, novel therapeutics and treatments are urgently needed that can safely and effective treat SHTG, in particular that can treat SHTG and diabetes at the same time.

### SUMMARY OF THE INVENTION

The invention is based in part on novel compositions and methods of use of BUDCA for treating SHTG, in particular for treating SHTG along with diabetes.

In one aspect, the invention generally relates to a method for treating SHTG, comprising administering to a subject in need thereof a therapeutically effective amount of a pharmaceutical composition comprising BUDCA.

In another aspect, the invention generally relates to a method for treating SHTG and diabetes (e.g., T2DM), comprising administering to a subject in need thereof a therapeutically effective amount of a pharmaceutical composition comprising BUDCA.

In another aspect, the invention generally relates to a method for treating SHTG and fatty liver diseases (e.g., NAFLD, NASH), comprising administering to a subject in need thereof a therapeutically effective amount of a pharmaceutical composition comprising BUDCA.

In yet another aspect, the invention generally relates to a unit dosage form of BUDCA comprising from about 750 mg to about 1,250 mg.

In yet another aspect, the invention generally relates to use of BUDCA for treating SHTG.

In yet another aspect, the invention generally relates to use of BUDCA for treating a subject with both SHTG and diabetes (e.g., T2DM).

In yet another aspect, the invention generally relates to use of BUDCA for treating a subject with SHTG or with both SHTG and diabetes (e.g., T2DM) who is unable to take or tolerate statin.

In yet another aspect, the invention generally relates to use of BUDCA as an adjuvant in treatment of SHTG or with both SHTG and diabetes (e.g., T2DM).

### BRIEF DESCRIPTION OF THE DRAWINGS

FIG. 1 shows certain exemplary information regarding changes from baseline to week 18 in LDL-C and Triglycerides.

FIG. 2 shows certain exemplary information regarding changes from baseline in HbA1c and fasting plasma glucose. The p-values were obtained from an ANCOVA model with treatment group as a fixed effect and baseline value of associated parameters as covariates.

FIG. 3 shows certain exemplary information regarding changes from baseline in body weight. The p-values were obtained from an ANCOVA model with treatment group as a fixed effect and baseline value of associated parameters as covariates.

FIG. 4 shows certain exemplary information regarding percent changes from baseline to end of treatment in triglycerides in subjects with HTG.

FIG. 5 shows certain exemplary information regarding percent changes from baseline to end of treatment in LDL-C and HDL-C in subjects with HTG.

FIG. 6 shows certain exemplary information regarding changes from baseline to end of treatment in HbA1c and fasting plasma glucose in subjects with HTG.

FIG. 7 shows certain exemplary information regarding changes from baseline to end of treatment in body weight in subjects with HTG. Changes in body weight were based solely on Study B as no post baseline measurement were performed in Study A.

FIG. 8 shows a schematic of a proposed phase 2 efficacy and safety study of BUDCA in adult subjects with SHTG.

### DEFINITIONS

Unless defined otherwise, all technical and scientific terms used herein have the same meaning as commonly understood by one of ordinary skill in the art to which this invention belongs. General principles of organic chemistry, as well as specific functional moieties and reactivity, are described in “Organic Chemistry”, Thomas Sorrell, University Science Books, Sausalito: 2006.

As used in the present disclosure, the following words and phrases are generally intended to have the meanings as set forth below unless expressly indicated otherwise or the context in which they are used indicates otherwise.

In this specification and the appended claims, the singular forms “a,” “an,” and “the” include plural reference, unless the context clearly dictates otherwise.

The term “and/of” is used in this disclosure to mean either “and” or “or” unless the context clearly dictates otherwise.

As used herein, “at least” a specific value is understood to be that value and all values greater than that value.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The term “comprising”, when used to define compositions and methods, is intended to mean that the compositions and methods include the recited elements, but do not exclude other elements. The term “consisting essentially of”, when used to define compositions and methods, shall mean that the compositions and methods include the recited elements and exclude other elements of any essential significance to the compositions and methods. For example, “consisting essentially of” refers to administration of the pharmacologically active agents expressly recited and excludes pharmacologically active agents not expressly recited. The term consisting essentially of does not exclude pharmacologically inactive or inert agents, e.g., pharmaceutically acceptable excipients, carriers or diluents. The term “consisting of”, when used to define compositions and methods, shall mean excluding trace elements of other ingredients and substantial method steps. Embodiments defined by each of these transition terms are within the scope of this invention.

In the application, where an element or component is said to be included in and/or selected from a list of recited elements or components, it should be understood that the element or component can be any one of the recited elements or components, or the element or component can be selected from a group consisting of two or more of the recited elements or components.

Where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

At various places in the present specification, variables or parameters are disclosed in groups or in ranges. It is specifically intended that the description include each and every individual subcombination of the members of such groups and ranges. For example, a range of 1 to 16 is understood to include any number, combination of numbers, or sub-range from the group consisting 1, 2, 3, 4, 5, 6, 7, 8, 9, 10, 11, 12, 13, 14, 15 or 16.

As used herein, the term “effective amount” of an active agent refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the patient.

As used herein, the term “treating”, “reducing”, or “preventing” a disease or disorder” refers to ameliorating such a condition before or after it has occurred. As compared with an equivalent untreated control, such reduction or degree of prevention is at least 5%, 10%, 20%, 40%, 50%, 60%, 80%, 90%, 95%, or 100% as measured by any standard technique.

As used herein, “pharmaceutical composition” refers to the combination of a therapeutically active agent with one or more pharmaceutically acceptable excipients, carriers, or diluents, making the composition especially suitable for diagnostic or therapeutic use in vivo or ex vivo.

As used herein, the term “pharmaceutically acceptable excipient, carrier, or diluent” refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

As used herein, the term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject. A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

As used herein, “administering” means oral administration, administration as a pulmonary, suppository, intramuscular administration, intrathecal administration, intranasal administration or subcutaneous administration, or the implantation of a slow-release device, e.g., a mini-osmotic pump, to a subject. Administration is by any route, including transmucosal (e.g., buccal, sublingual, palatal, gingival, nasal, vaginal, rectal, or). Parenteral administration includes, e.g., intramuscular and subcutaneous. Other modes of delivery include, but are not limited to, the use of liposomal formulations, etc. By “co-administer” it is meant that a composition described herein is administered at the same time, just prior to, or just after the administration of one or more additional therapies (e.g., therapeutic agent, chemotherapeutic, or treatment for a neurodegenerative disease). The compound of formula (I) can be administered alone or can be co-administered to the patient. Co-administration is meant to include simultaneous or sequential administration of the compound individually or in combination (more than one compound or agent). Thus, the preparations can also be combined, when desired, with other active substances (e.g., to reduce metabolic degradation).

The terms “disease,” “disorder,” and “condition” are used interchangeably herein.

As used herein, the term “treating”, “reducing”, or “preventing” a disease or disorder refers to ameliorating such a condition before or after it has occurred. As compared with an equivalent untreated control, such reduction or degree of prevention is at least 5%, 10%, 20%, 40%, 50%, 60%, 80%, 90%, 95%, or 100% as measured by any standard technique. The terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”). In one embodiment, the compounds provided herein are contemplated to be used in methods of therapeutic treatment wherein the action occurs while a subject is suffering from the specified disease, disorder or condition and results in a reduction in the severity of the disease, disorder or condition, or retardation or slowing of the progression of the disease, disorder or condition. In an alternate embodiment, the compounds provided herein are contemplated to be used in methods of prophylactic treatment wherein the action occurs before a subject begins to suffer from the specified disease, disorder or condition and results in preventing a disease, disorder or condition, or one or more symptoms associated with the disease, disorder or condition, or preventing the recurrence of the disease, disorder or condition.

### DETAILED DESCRIPTION OF THE INVENTION

The invention is based in part on compositions and methods of use of BUDC for treating patients with SHTG and/or with SHTG and DM. In particular, the present invention relates to a novel strategy for treating SHTG and in particular for treating SHTG patients that are also diabetic.

BUDC is an ionic salt of BBR and UDCA, represented by

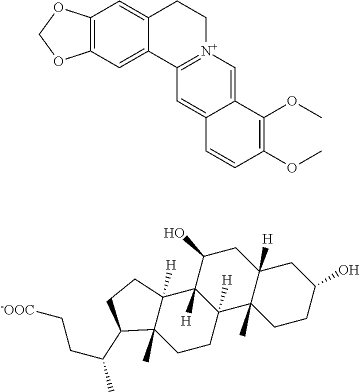

### Berberine Ursodeoxycholate (BUDC)

The compound was disclosed in WO 2016/015634 A1 (PCT/CN2015/085350) and WO 2018/205987 A1 (PCT/CN2018/086461), the content of each of which is incorporated herein by reference in its entirety.

BUDC (also referred to as BUDCA) is under investigation for the treatment of primary sclerosing cholangitis (PSC), primary biliary cirrhosis (PBC) and non-alcoholic steatohepatitis (NASH). BUDC is an ionic salt formed between BBR and UDCA with 1:1 stoichiometry and unique physico-chemical properties including enhanced bioavailability of BBR within the gut.

Efficacy demonstrated previously in non-clinical and clinical studies of BUDC include: improving glucose homeostasis, positively regulating lipoprotein metabolism, attenuating cholestatic liver and bile duct injury, anti-inflammatory, anti-fibrotic actions, and beneficial effects on gut microbiome.

In one aspect, the invention generally relates to a method for treating SHTG, comprising administering to a subject in need thereof a therapeutically effective amount of a pharmaceutical composition comprising BUDCA.

In another aspect, the invention generally relates to a method for treating SHTG and diabetic disease (DM), comprising administering to a subject in need thereof a therapeutically effective amount of a pharmaceutical composition comprising BUDCA.

In certain embodiments, the diabetic disease is T2DM.

In another aspect, the invention generally relates to a method for treating SHTG and a fatty liver diseases, comprising administering to a subject in need thereof a therapeutically effective amount of a pharmaceutical composition comprising BUDCA.

In certain embodiments, the fatty liver disease is metabolic dysfunction-associated fatty liver disease (MAFLD) or non-alcoholic fatty liver disease (NAFLD).

In certain embodiments, the fatty liver disease is nonalcoholic steatohepatitis (NASH).

In certain embodiments, BUDCA is administered as a monotherapy.

In certain embodiments, the subject is also administered one or more other therapy/therapies in addition to BUDCA. In certain embodiments, the subject is also administered one or more of metformin, insulin and a GLP-1 agonist.

In certain embodiments, BUDCA is administered as an adjuvant.

In certain embodiments, the subject is unable to take or tolerate statin.

In certain embodiments, BUDCA is administered BID at about 750 mg to about 1,250 mg per administration.

In certain embodiments, BUDCA is in the form of Form A. In certain embodiments, BUDCA is a hydrate (e.g., a hemi-nonahydrate).

In another aspect, the invention generally relates to a unit dosage form of BUDCA (e.g., comprising from about 750 mg to about 1,250 mg). In certain embodiments, the unit dosage form comprises a pharmaceutically acceptable excipient, carrier, or diluent

In certain embodiments of the unit dosage form, BUDCA is in the form of Form A.

In certain embodiments of the unit dosage form, BUDCA is a hydrate (e.g., a hemi-nonahydrate).

In certain embodiments, the unit dosage form is capsule.

In certain embodiments, the unit dosage form is tablet.

In yet another aspect, the invention generally relates to use of BUDCA for treating SHTG.

In yet another aspect, the invention generally relates to use of BUDCA for treating a subject with both SHTG and DM.

In yet another aspect, the invention generally relates to use of BUDCA for treating a subject with both SHTG and T2DM.

In yet another aspect, the invention generally relates to use of BUDCA for treating a subject with SHTG or with both SHTG and DM who is unable to take or tolerate statin.

In yet another aspect, the invention generally relates to use of BUDCA for treating a subject with both SHTG and fatty liver disease.

In yet another aspect, the invention generally relates to use of BUDCA for treating a subject with both SHTG and NAFLD.

In yet another aspect, the invention generally relates to use of BUDCA for treating a subject with both SHTG and NASH.

In yet another aspect, the invention generally relates to use of BUDCA for treating a subject with SHTG or with both SHTG and fatty liver disease who is unable to take or tolerate statin.

In certain embodiments of the methods disclosed herein, BUDC is administered at a daily dosage in the range of about 250 mg to about 5,000 mg (e.g., about 500 mg to about 3,000 mg, about 750 mg to about 3,000 mg, about 1,000 mg to about 3,000 mg, about 1,500 mg to about 3,000 mg, about 250 mg to about 2,000 mg, about 250 mg to about 1,500 mg, about 250 mg to about 1,000 mg, about 500 mg to about 1,500 mg, about 750 mg to about 1,250 mg) for a time period of about 1 day to about 24 weeks (e.g., about 1 day to about 12 weeks, about 1 day to about 6 weeks, about 1 day to about 4 weeks, about 1 day to about 2 weeks, about 1 week to about 24 weeks, about 4 weeks to 24 weeks, about 6 weeks to 24 weeks) or longer.

In certain embodiments, the subject is administered from about 750 mg to about 1,250 mg BID of BUDCA.

In certain embodiments, the pharmaceutical composition of BUDC disclosed herein comprises a pharmaceutically acceptable excipient, carrier, or diluent.

Various solid and crystalline forms of BUDC may be employed in the invention disclosed herein.

In certain embodiments, Form A of BUDC, having an X-ray powder diffraction (XRPD) pattern comprising one or more peaks at 2θ values selected from the group consisting of: 3.98, 7.06, 7.34, 7.93, 8.79, 9.47, 11.70, 11.94, 12.34, 12.55, 13.90, 14.17, 15.14, 15.50, 16.16, 16.54, 16.78, 17.53, 17.67, 18.23, 19.03, 19.98, 20.87, 21.13, 21.96, 23.49, 24.24, 24.97, 25.50, 26.63, 27.60, 28.06, 28.63, 29.40 and 30.49° (±0.2°) obtained using Cu Kα radiation (λ₁=1.540598 Å, λ₂=1.544426 Å, intensity ratio Δ₂/λ₁=0.50), is employed.

In certain embodiments, Form B of BUDC, having an X-ray powder diffraction (XRPD) pattern comprising one or more peaks at 2θ values selected from the group consisting of: 7.39, 9.31, 12.41, 13.14, 14.37, 14.76, 15.53, 18.65, 21.79, 22.87, 25.27, 25.53 and 28.12° (0.2°) obtained using Cu Kα radiation (λ₁=1.540598 Å, λ₂=1.544426 Å, intensity ratio λ₂/λ₁=0.50), is employed.

In certain embodiments, Form C of BUDC, having an X-ray powder diffraction (XRPD) pattern comprising one or more peaks at 2θ values selected from the group consisting of: 7.23, 10.42, 12.10, 13.37, 14.24, 14.48, 15.28, 15.95, 17.00, 18.17, 20.12, 21.77 and 25.47° (±0.2°) obtained using Cu Kα radiation (λ₁=1.540598 Å, λ₂=1.544426 Å, intensity ratio λ₂/λ₁=0.50), is employed.

In certain embodiments, Form D of BUDC, having an X-ray powder diffraction (XRPD) pattern comprising one or more peaks at 2θ values selected from the group consisting of: 4.24, 6.79, 8.50, 10.25, 11.50, 13.62, 14.74, 15.20, 17.92, 18.39, 22.91 and 25.73° (±0.2°) obtained using Cu Kα radiation (λ₁=1.540598 Å, λ₂=1.544426 Å, intensity ratio)₂/λ₁=0.50), is employed.

In certain embodiments, Form E of BUDC, having an X-ray powder diffraction (XRPD) pattern comprising one or more peaks at 2θ values selected from the group consisting of: 8.59, 10.55, 11.36, 11.86, 12.46, 13.08, 13.38, 14.34, 15.57, 17.24, 17.72, 18.43, 19.66, 19.84, 20.35, 20.91, 21.36, 21.95, 23.21, 24.67, 25.04, 25.82, 26.12, 27.01, 27.84, 28.97, 30.35, 33.33, 34.54 and 36.06° (±0.2°) obtained using Cu Kα radiation (λ₁=1.540598 Å, λ₂=1.544426 Å, intensity ratio λ₂/λ₁=0.50), is employed.

In certain embodiments, Form H of BUDC, having an X-ray powder diffraction (XRPD) pattern comprising one or more peaks at 2θ values selected from the group consisting of: 13.05, 14.63 and 25.46° (±0.2°) obtained using Cu Kα radiation (λ₁=1.540598 Å, λ₂=1.544426 Å, intensity ratio λ₂/λ₁=0.50), is employed.

In certain embodiments, Form I of BUDC, having an X-ray powder diffraction (XRPD) pattern comprising one or more peaks at 2θ values selected from the group consisting of: 4.19, 7.64, 10.03, 13.32, 13.84, 14.83, 16.73, 22.73, 25.61 and 28.57° (0.2°) obtained using Cu Kα radiation (λ₁=1.540598 Å, λ₂=1.544426 Å, intensity ratio λ₂/λ₁=0.50), is employed.

In certain embodiments, Form J of BUDC, having an X-ray powder diffraction (XRPD) pattern comprising one or more peaks at 2θ values selected from the group consisting of: 4.61, 6.32, 7.38, 8.22, 9.21, 10.57, 11.73, 12.13, 12.62, 12.96, 13.87, 14.55, 14.78, 15.81, 16.48, 17.69, 18.39, 19.01, 20.06, 21.25, 22.13, 23.20, 24.47, 24.89, 26.31, 27.98, 30.25 and 33.35° (±0.2°) obtained using Cu Kα radiation (λ₁=1.540598 Å, λ₂=1.544426 Å, intensity ratio λ₂/λ₁=0.50), is employed.

In certain embodiments, Form P of BUDC, having an X-ray powder diffraction (XRPD) pattern comprising one or more peaks at 2θ values selected from the group consisting of: 3.11, 5.01, 5.78, 7.26, 9.20, 10.10, 10.79, 11.65, 13.70, 14.59, 15.22, 16.19, 16.54, 17.05, 18.06, 18.68, 20.52, 21.09, 21.73, 22.49, 24.73, 25.42, 25.94 and 30.11° (±0.2°) obtained using Cu Kα radiation (λ₁=1.540598 Å, λ₂=1.544426 Å, intensity ratio λ₂/λ₁=0.50), is employed.

In certain embodiments, Form W of BUDC, having an X-ray powder diffraction (XRPD) pattern comprising one or more peaks at 2θ values selected from the group consisting of: 6.49, 7.16, 8.51, 10.21, 12.01, 13.13, 13.90, 14.42, 15.18, 15.57, 16.03, 16.45, 16.74, 17.08, 17.85, 18.39, 19.61, 20.43, 21.39, 21.70, 23.51 and 25.21° (0.2°) obtained using Cu Kα radiation (λ₁=1.540598 Å, λ₂=1.544426 Å, intensity ratio)₂/λ₁=0.50), is employed.

In certain embodiments, Form X of BUDC, having an X-ray powder diffraction (XRPD) pattern comprising one or more peaks at 2θ values selected from the group consisting of: 3.63, 6.61, 7.24, 10.49, 11.95, 13.51, 14.26, 14.54, 15.14, 16.01, 16.82, 18.28, 20.26, 21.08, 21.49, 21.90, 25.60, 26.40, 27.31, 29.34, 30.59, 31.01, 34.04, 34.68 and 36.91° (0.2°) obtained using Cu Kα radiation (λ₁=1.540598 Å, λ₂=1.544426 Å, intensity ratio λ₂/λ₁=0.50), is employed.

In certain embodiments, the solid or crystalline form (e.g., Form A of BUDC) is a hydrate of BUDC. In certain embodiments, the solid or crystalline form (e.g., Form A of BUDC) is a hemi-nonahydrate of BUDC.

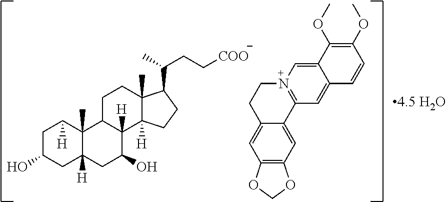

### Hemi-Nonahydrate of BUDC

In certain embodiments, the solid or crystalline form (e.g., Form A of BUDC) is crystalline. In certain embodiments, the crystalline form is characterized in a monoclinic crystal system and P2₁ space group. In certain embodiments of the crystalline form, each unit cell contains two asymmetric units and there are two BBR cations, two UDCA anions and nine H₂O molecules per asymmetric unit, and four BBR cations, four UDCA anions and eighteen H₂O molecules per unit cell.

Possible formulations include those suitable for oral, sublingual, buccal, parenteral (for example subcutaneous, intramuscular, or intravenous), rectal, topical including transdermal, intranasal and inhalation administration. Most suitable means of administration for a particular patient will depend on the nature and severity of the disease or condition being treated or the nature of the therapy being used and on the nature of the active compound.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

The following examples are meant to be illustrative of the practice of the invention, and not limiting in any way.

### EXAMPLES

Various pre-clinical and clinical studies were performed to evaluate safety and efficacy of BUDCA. Primary and secondary pharmacodynamics studies were performed. Specific to nonclinical proof of concept for BUDCA in SHTG, the efficacy of BUDCA on hyperlipidemia/NAFLD was evaluated in two studies (Study A and Study B) using a high fat diet induced hyperlipidemia/NAFLD hamster model. Dietary induced changes in hamsters can be considered a valid model for dyslipidemia.

### Example 1. Nonclinical Pharmacology—Study A

Male Golden Syrian hamsters were fed with a high-fat/high-cholesterol diet which contained 1.25% cholesterol, 20.06% grease (2.79% soybean oil, 17.27% cocoa butter) to establish the hyperlipidemia/NAFLD model. After 2 weeks of high-fat/high-cholesterol diet inducement, 24 hamsters with fasting TC level 17.96±1.70 mmol/L were selected and divided into 3 groups of 8 hamsters each (model group G1, 50 mg/kg/day BUDCA group G2, and 200 mg/kg/day BUDCA group G3). Eight hamsters comprising the normal control were fed with a normal chow diet.

The normal and Model control groups received vehicle (10 mL/kg 0.5% gum tragacanth solution), while the two BUDCA treatment groups received BUDCA, via intragastric administration for 7 weeks prior to sacrifice.

During the treatment period, blood was collected at weeks 1, 3, 5, and 7, for biochemical analysis including serum TC, TG, HDL-c, LDL-c, and liver enzymes. At the end of the study, animals were sacrificed for liver macroscopic observation and liver tissues were collected for histopathological evaluations and liver lipid contents.

### Effects of BUDCA on Serum Lipids (TC, TG, HDL-c and LDL-c)

As shown in Table 1, compared to the normal control group (G0), the serum TG, TC, HDL-c, LDL-c levels, and the atherosclerosis index (AI) of the model group (G1) significantly increased (P<0.01). Compared to the model group, treatment with BUDCA for 7 weeks significantly reduced serum TG, TC, LDL-c levels, and AI (P<0.01) in a dose-dependent manner. Noteworthy is that the serum lipid levels of TG, TC, HDL-c, LDL-c, and AI in the high dose BUDCA treatment group reduced lipid levels to those of the normal control. In addition, 7-weeks of BUDCA treatment significantly reduced the serum PCSK9 levels (P<0.05) compared to the Model control group suggesting that BUDCA inhibition of PCSK9 may in part serve as a mechanism underlying its beneficial effect on lipid metabolism.

TABLE 1
Effects of BUDCA on lipids and PCSK9 after 7 weeks treatment (mean ± SEM)

| | | TG | TC | LDL-c | HDL-c | AI | PCSK9 |
|---|---|---|---|---|---|---|---|
| Group | Dosage | (mmol/L) | (mmol/L) | (mmol/L) | (mmol/L) | value | (pg/mL) |
| Normal (G0) | Vehicle | 1.07 ± 0.20 | 4.05 ± 0.33 | 1.30 ± 0.21 | 1.96 ± 0.17 | 1.07 ± 0.15 | 50.9 ± 6.1 |
| Model (G1) | Vehicle | 4.79 ± 2.21ΔΔ | 21.74 ± 6.44ΔΔ | 8.11 ± 2.39ΔΔ | 3.48 ± 0.29ΔΔ | 5.39 ± 2.36ΔΔ | 403.1 ± 73.9ΔΔΔ |
| Low dose | 50 mg/kg/day | 3.88 ± 1.21 | 9.78 ± 2.58** | 2.95 ± 1.72** | 3.42 ± 0.28 | 1.88 ± 0.83** | 124.9 ± 16.8** |
| group (G2) | BUDCA | | | | | | |
| High dose | 200 mg/kg/day | 1.90 ± 0.66** | 4.95 ± 0.84** | 1.23 ± 0.28** | 2.53 ± 0.29** | 0.95 ± 0.19** | 179.0 ± 30.9* |
| group (G3) | BUDCA | | | | | | |

Note:
1) As compared with the normal control group,
ΔP < 0.05,
ΔΔP < 0.01;
as compared with the model control group,
*P < 0.05,
**P < 0.01.
2) Atherosclerosis index (AI) value is a risk indicator in lipid metabolism disorders, it's value for each animal was calculated as AI = (TC − HDL-c)/HDL-c.

### Effects of BUDCA on Liver Contents of Triglycerides and Total Cholesterol

As shown in Table 2, compared to the normal group, the TG and TC contents in liver were significantly increased in the model group (P<0.01). However, following 7 weeks of treatment with BUDCA, liver contents of TG and TC were significantly decreased in the BUDCA treatment groups (both doses), when compared to the model group (P<0.01). Treatment with both doses of BUDCA reversed the contents of liver TG to that observed in normal control group.

TABLE 2
Effects of BUDCA on Liver TG and TC Contents

| Group | Dosage | Liver TG (mg/g) | Liver TC (mg/g) |
|---|---|---|---|
| Normal (G0) | Vehicle | 6.36 ± 2.07 | 2.28 ± 0.69 |
| Model (G1) | Vehicle | 31.04 ± 9.62^{ΔΔ} | 33.99 ± 7.18^{ΔΔ} |
| Low dose group (G2) | 50 mg/kg/day BUDCA | 5.40 ± 2.64** | 14.62 ± 4.89** |
| High dose group (G3) | 200 mg/kg/day BUDCA | 5.61 ± 1.98** | 3.82 ± 1.78** |

Note:
Compared with the normal control group:
^{Δ}P < 0.05,
^{ΔΔ}P < 0.01;
Compared with the model group:
*P < 0.05,
**P < 0.01

### Example 2. Nonclinical Pharmacology—Study B

Study B was conducted using the same hamster model as described in Study A to evaluate the efficacy of BUDCA as well as to compare the effects of BUDCA with equivalent doses of either UDCA or BBR-Cl on their own. The model was induced with the same method as indicated in Study A. After the 2-week induction period, hamsters were randomized into treatment groups and continued the high fat diet for an additional 6-week period, concurrent with the daily oral administration of various doses of BUDCA (Low dose: 50 mg/mL; Medium dose: 100 mg/mL; High dose: 200 mg/mL), BBR-Cl on its own (Low dose: 51.1 mg/mL; High dose: 102.2 mg/mL), UDCA on its own (Low dose: 53.9 mg/mL; High dose: 107.8 mg/mL), or a 1:1 physical mixture of BBR-Cl and UDCA equivalent to a lower dose of BUDCA (105.0 mg/mL). A model control group continued the high fat diet and normal control group fed the standard diet were both administered the vehicle 0.5% carboxymethyl cellulose sodium (CMC-Na) solution. All animals were sacrificed for terminal evaluations at the end of the 6-week dosing period.

Table 3 shows results from high dose of BUDCA (200 mg/kg) and the equimolar content of each individual component, UDCA or BBR-Cl.

TABLE 3
Representative Study Groups for Data Presentation (High Dose)

| | | Volume | Number in |
|---|---|---|---|
| Group | Dose | (mL/kg) | each group |
| Normal control | VH¹: 10 mL/kg | 10 | 8 |
| | 0.5% CMC-Na solution | | |
| Model control | VH: 10 mL/kg | 10 | 8 |
| | 0.5% CMC-Na solution | | |
| BUDCA | 200 mg/kg BUDCA | 10 | 8 |
| UDCA | 107.8 mg/kg UDCA | 10 | 8 |
| BBR-Cl | 102.2 mg/kg BBR-Cl | 10 | 8 |

Note:
For BBR-Cl and UDCA, doses are equimolar to the content of each individually in the corresponding dose of BUDCA.
¹VH = vehicle

Endpoints evaluated included serum lipids (TC, TG, LDL-C, HDL-C), liver lipid content (TC and TG), liver injury (ALT, AST, total bile acids (TBA) and total bilirubin (TBIL)), liver weight (expressed relative to body weight as liver index), macroscopic observation and histopathology evaluation of the livers. Histopathology endpoints included pathologist observation, as well as numerical comparison via the nonalcoholic fatty liver disease activity score (NAS) and a fibrosis severity score.

The hyperlipidemia/NAFLD golden hamster model resulted in a significant increase in serum TC, TG, LDL-C, HDL-C, ALT, AST, TBA, and TBIL levels. Terminal endpoints in model control animals also confirmed increased liver weight and histopathology of the liver as evidenced by steatosis, hepatocytes swelling, punctate necrosis, inflammatory cell infiltration and local fibrosis. Therefore, the model was not only able to replicate key characteristics of the NAFLD disease spectrum in humans, but also hyperlipidemia.

Relative to the model control animals, BUDCA administration reduced all the following parameters in a dose dependent manner: serum TC and TG, LDL-C, ALT, AST, TBA, TBIL, liver weight, liver TC and TG, NAS score, and fibrosis (score and % area). Treatment with BUDCA was observed to completely reverse the effect of the high fat diet on multiple clinically relevant features of hyperlipidemia and NASH.

Taken together, the data demonstrate that in a hamster model of hyperlipidemia/NAFLD, BUDCA administration (1) has a significant hypolipidemic effect and can reduce both serum lipids and lipid deposition in the liver; and (2) reduces liver injury as indicated by serum markers (AST, AST, TBIL, TBA) and histological evidence of steatosis, inflammation, ballooning degeneration, and fibrosis. These effects are dose dependent and greatest at the high dose of 200 mg/kg BUDCA, where several models induced, changes were returned to a state similar to normal animals.

Importantly, the beneficial effects of BUDCA on most of these parameters, including serum lipids and liver lipid deposition, were greater than those achieved with either UDCA or BBR when administered alone. While improvements in some parameters were also observed with UDCA and/or BBR-Cl on their own, the magnitude was not to the extent as the equivalent dose of BUDCA, demonstrating improved performance of BUDCA as a single active ingredient over the individual moieties UDCA and BBR.

In regard to BUDCA for treatment of SHTG, results on serum lipids and liver lipid deposition data are shown in Table 4 and Table 5, respectively.

TABLE 4
Effects of BUDCA on Serum Lipids of TG, TC, LDL-
and HDL-e after 6 weeks treatment (mean ± SEM)

| | | Serum lipids | | | |
|---|---|---|---|---|---|
| | | TG | TC | LDL-c | HDL-c |
| Group | Dose | (mmol/L) | (mmol/L) | (mmol/L) | (mmol/L) |
| Normal control | ¹VH | 1.41 ± 0.18 | 3.72 ± 0.32 | 1.40 ± 0.20 | 2.03 ± 0.06 |
| group | | | | | |
| Model control | ¹VH | 3.60 ± 0.55^{##} | 18.99 ± 2.18^{##} | 7.89 ± 1.03^{##} | 5.03 ± 0.21^{##} |
| group | | | | | |
| BUDCA | 200 mg/kg BUDCA | 2.06 ± 0.39** | 7.03 ± 0.73** | 1.26 ± 0.15** | 4.25 ± 0.40* |
| UDCA | 107.8 mg/kg UDCA | 2.17 ± 0.18** | 10.13 ± 0.73** | 3.83 ± 0.47** | 4.81 ± 0.15 |
| BBR-Cl | 102.2 mg/kg BBR-Cl | 3.72 ± 0.37 | 14.86 ± 1.23* | 6.53 ± 1.10 | 5.24 ± 0.30 |

Note:
1) For BBR-Cl and UDCA, doses are equimolar to the content of each individually in the
corresponding dose of BUDCA.
¹VH = vehicle;
2) Compared with the normal control group,
^{#}P < 0.05,
^{##}P < 0.01;
Compared with the model control group,
*P < 0.05,
**P < 0.01.

TABLE 5
Effects on Liver Lipid Deposition (mean ± SEM)

| | | Liver Lipid Deposition | | | |
|---|---|---|---|---|---|
| | | | | Steatosis | |
| | | | | Score of | Proportion of oil |
| | | Liver TG | Liver TC | HE | red O staining |
| Group | Dose | (mg/g) | (mg/g) | staining | positive area (%) |
| Normal control | VH | 4.88 ± 0.70 | 1.89 ± 0.14 | 0.00 ± 0.00 | 0.05 ± 0.01 |
| group | | | | | |
| Model control | VH | 15.38 ± 2.23^{##} | 30.95 ± 4.41^{##} | 2.88 ± 0.13^{##} | 30.30 ± 1.89^{##} |
| group | | | | | |
| BUDCA | 200 mg/kg BUDCA | 3.92 ± 0.50** | 4.59 ± 0.84** | 1.00 ± 0.38** | 1.18 ± 0.69** |
| UDCA | 107.8 mg/kg UDCA | 11.24 ± 2.69 | 17.95 ± 4.04* | 2.13 ± 0.35* | 11.72 ± 4.65** |
| BBR-Cl | 102.2 mg/kg BBR-Cl | 19.18 ± 2.24 | 29.27 ± 4.47 | 2.75 ± 0.16 | 18.97 ± 2.43* |

Note:
1) For BBR-Cl and UDCA, doses are equimolar to the content of each individually in the corresponding dose of BUDCA.
¹VH = vehicle;
2) Compared with the normal control group,
^{#}P < 0.05,
^{##}P < 0.01;
Compared with the model control group,
*P < 0.05,
**P < 0.01.

Efficacy was also tested in two clinical studies that established merits for further clinical study in patients with SHTG: Study C was conducted in adults with hypercholesterolemia and Study C was conducted in adults with NASH and T2DM. A pooled analysis was conducted based on these two studies specifically evaluating the data from subjects who had HTG at baseline (TG 200 mg/dL).

### Example 3. Clinical Study in Subjects with Hypercholesteremia—Study C

In a Phase 1b/2a study, 50 overweight or obese adults with hypercholesterolemia were enrolled and treated with BUDCA for 28 days. The primary objective was to evaluate safety and tolerability of BUDCA. Given that the subjects enrolled in this study were obese with a history of hypercholesteremia, the focus of the efficacy and PD endpoints was lipid metabolism. Key entry criteria included BMI 2 and LDL-C, BUDCA 250 mg BID (N=12), BUDCA 500 mg BID (N=12), or BUDCA 1000 mg BID (N=12).

While all subjects had hypercholesteremia at baseline, TG levels generally ranged from normal to mild HTG (three subjects from each of placebo, BUDCA 500 mg BID, and BUDCA 1000 mg BID had TG values >200 mg/dL) and HDL-C levels were generally in the normal range.

Among subjects receiving BUDCA, serum lipid levels decreased in a dose-dependent fashion, with maximum reductions relative to placebo being observed after 14 days of treatment. Statistically significant differences were observed with BUDCA at various dose levels compared with placebo for TGs, TC, LDL-C, lipoprotein-a, and non-HDL-C. The statistical differences observed for TGs and VLDL-C were largely driven by worsening from baseline with placebo.

### Example 4. Clinical Study in Subjects with NASH and T2DM—Study D

Study D was a randomized, double-blind, placebo-controlled Phase 2 study in adults with NASH and T2DM. The primary objective of this study was to evaluate the change from baseline in liver fat content as assessed by MRI-PDFF, but also evaluated changes in key cardiometabolic parameters, including lipids. Key entry criteria included the clinical diagnosis of NASH and T2DM and BMI >25.0 kg/m2. Subjects were randomized and treated with placebo (N=33), BUDCA 500 mg BID (N=33), or BUDCA 1000 mg BID (N=34) for 18 weeks (Harrison 2021).

Demographic characteristics were generally well balanced among treatment groups. The median age overall was 57 years (range: 26 to 75 years), and there were 72% females and 28% males. Most subjects were White (91%) and not Hispanic or Latino (62%). Mean baseline weight and BMI were similar across treatment groups. On average, LDL-C was elevated and near the upper limit of the normal range and TGs were elevated above the normal. Baseline values for HbA1c, ALT, GGT, liver fat content, and cT1 were similar across treatment groups.

Study D evaluated the effects of BUDCA on lipids as secondary efficacy endpoints. On average, subjects had near-normal LDL-C at baseline with borderline-high levels of TG. While the average values were generally similar across treatment groups, it should be noted that LDL-C was lower in the BUDCA 500 mg BID treatment group while TGs were lower in the BUDCA 1000 mg BID treatment group (Table 6). As can be seen in FIG. 1, TG levels were reduced with both doses of BUDCA (BUDCA 1000 mg BID: −11%; BUDCA 500 mg BID: −9%) in contrast to an increase with placebo (10%). Reductions in TGs with the lower 500 mg BID dose were statistically significant compared with placebo. Reduced TG levels were paralleled with a reduction in LDL-C after 18 weeks of treatment with BUDCA 1000 mg BID (−13%), but not with BUDCA 500 mg BID (3%) or with placebo (0%).

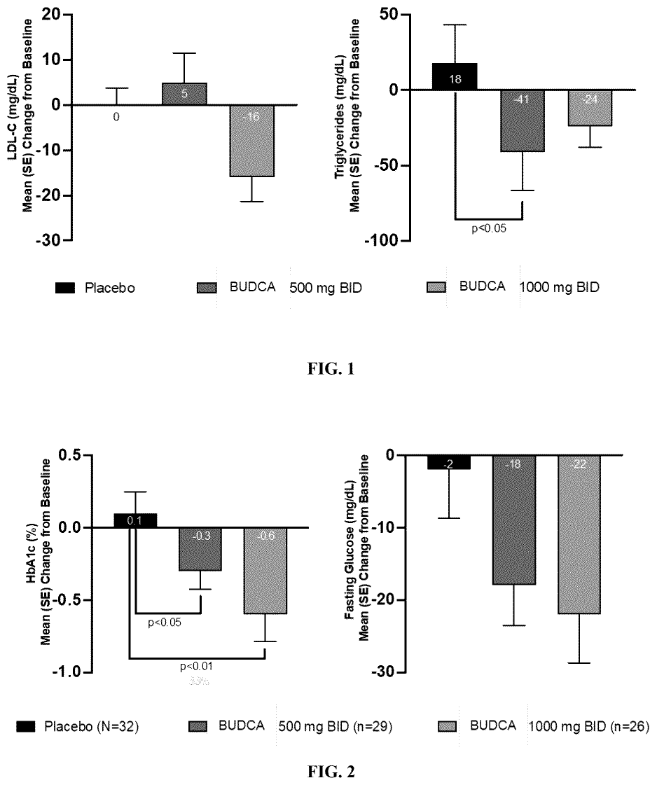

Significant dose dependent reductions were also observed with HbA1c with 18 weeks of BUDCA treatment in contrast to an increase observed in subjects who had been randomized to placebo as shown in FIG. 2. These data are further supported by a dose dependent numerical trend in fasting plasma glucose where in the mean change in fasting plasma glucose was −22 mg/dL, −18 mg/dL, and −2 mg/dL with BUDCA 1000 mg BID, BUDCA 500 mg BID and placebo, respectively.

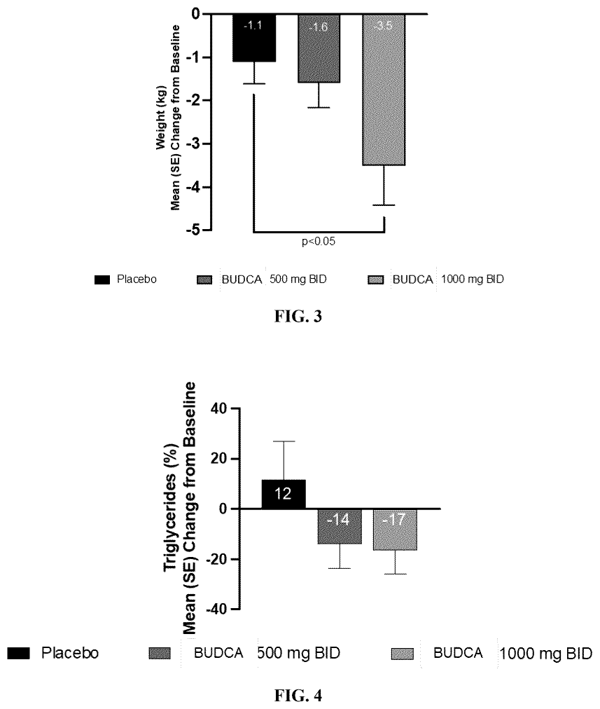

In addition to improvements observed in glucose metabolism, significant reductions were observed in body weight from baseline compared to placebo. As shown in FIG. 3, following 18 weeks of treatment, −3.5 kg vs −1.1 kg (p<0.05) reduction was observed with BUDCA 1000 mg BID compared to placebo.

To further investigate the potential of BUDCA for lowering TGs in patients with elevated TGs, a pooled analysis across studies Study C and Study D was conducted focusing on the subjects with baseline TGs above 200 mg/dL (HTG) (Table 6). The analysis pooled the baseline and end of treatment timepoints for this evaluation as the two studies had different durations (4 weeks vs 18 weeks).

TABLE 6
Pooled Analysis of Subjects with HTG

| | | BUDCA | |
|---|---|---|---|
| | Placebo | 500 mg BID | 1000 mg BID |
| Mean (SD) | (N = 16) | (N = 9) | (N = 10) |
| Study A (n/N) | 3/16 | 3/9 | 3/10 |
| Study B (n/N) | 13/16 | 6/9 | 7/10 |
| Triglycerides | 297.2 (105.88) | 397.6 (313.37) | 275.5 (65.53) |
| (mg/dL) | | | |
| LDL-C (mg/dL) | 101.8 (36.40) | 97.5 (55.02) | 116.1 (33.94) |
| HDL-C (mg/dL) | 35.1 (6.47) | 37.8 (6.48) | 43.6 (11.75) |
| HbA1c (%) | 6.6 (1.09) | 6.3 (1.05) | 6.5 (1.09) |
| Weight (kg) | 105.5 (26.96) | 84.7 (14.10) | 95.0 (18.83) |

At baseline, the TG levels were similar between placebo and BUDCA 1000 mg BID, but were further elevated and had a larger standard deviation in the subjects who had received BUDCA 500 mg BID. At the end of treatment, a reduction in TGs was observed with both doses of BUDCA compared with an increase in subjects who had received placebo as shown in FIG. 4. The placebo-corrected median percent change from baseline (95% CI) was −17.7% (−55.57, 16.61) and −12.3% (−52.63, 17.93) for subjects who had received BUDCA 1000 mg BID and BUDCA 500 mg BID, respectively. Of note, the study of Vascepa in subjects with HTG (the ANCHOR Study) showed similar TG reductions with a placebo-corrected median percent change of −21.5% and −10.1% in the high dose and low dose groups, respectively (Ballantyne 2012).

The further beneficial effects of BUDCA can be observed in the HTG population in the general improvement of key cardiometabolic parameters. Both doses of BUDCA resulted in a decrease in LDL-C at the end of treatment and an increase in HDL-C with BUDCA 1000 mg BID as shown in FIG. 5. In contrast, an increase in LDL-C was observed in placebo-treated subjects and no changes in HDL-C.

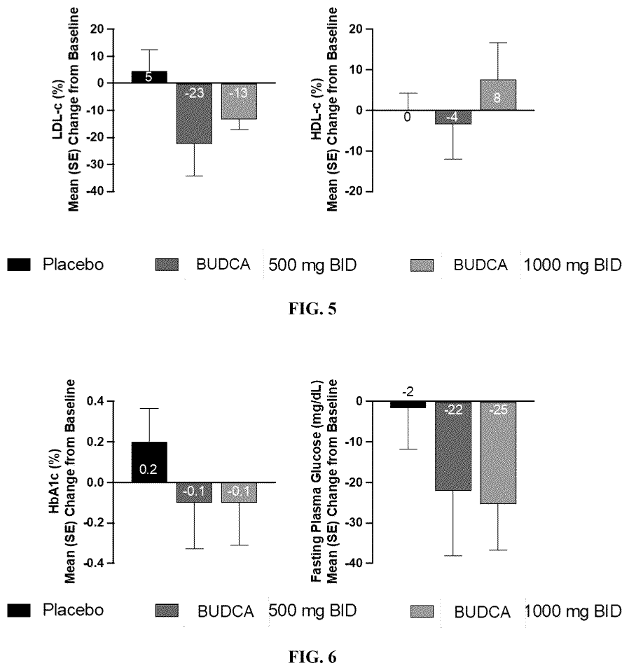

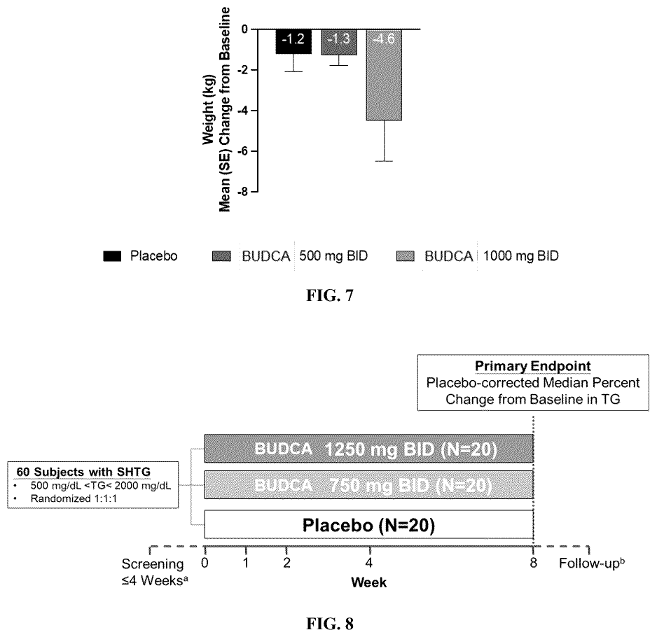

Further, in the HTG subgroup, both doses of BUDCA resulted in no worsening of HbA1c and a decrease in fasting plasma glucose while treatment with placebo resulted in a further increase in HbA1c and almost no change in fasting plasma glucose (FIG. 6). Lastly, as shown in FIG. 7, treatment of subjects with HTG with BUDCA 1000 mg BID resulted in a substantial reduction in body weight compared to placebo (−4.6 kg vs −1.2 kg).

### Example 5 (Prophetic): A Phase 2 Randomized, Double-Blind, Placebo-Controlled Study to Evaluate the Efficacy and Safety of BUDCA in Adult Subjects with Severe Hypertriglyceridemia (SHTG)

A Phase 2 randomized, double-blind, placebo-controlled dose-ranging study is carried out to evaluate the efficacy and safety of BUDCA in approximately 60 adult subjects with SHTG (see FIG. 8) and to study BUDCA's ability to reduce serum TG levels, which is an FDA-accepted and well-established marker for adult patients with SHTG. During the study, additional endpoints are evaluated that can help characterize the potential for broader metabolic and anti-inflammatory effects of BUDCA in SHTG.

Eligible subjects (500 mg/dL≤TG levels≤2000 mg/dL) are stratified by baseline TG level above and below 750 mg/dL and randomized 1:1:1 to receive BUDCA 1250 mg BID, BUDCA 750 mg BID or placebo for 8 weeks. Study visits occur at baseline (Day 0); Weeks 2, 4, 6, 8; and follow-up (4 weeks after the last dose).

To ensure a representative SHTG study population, subjects with a broad spectrum of metabolic disease (e.g., mixed dyslipidemia, T2DM and NAFLD) are included.

The primary endpoint analysis is carried out to evaluate the effects of BUDCA compared to placebo on TG levels. Secondary endpoints are also included to evaluate the effects of BUDCA as compared to placebo on other lipids including the changes in LDL-C, HDL-C, VLDL-C, and non-HDL-C.

BUDCA significantly reduces TG levels in study subjects with SHTG and such reduction in TG levels is greater than the reduction in subjects with HTG (TG<500).

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples disclosed herein are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. The above examples contain important additional information, exemplification and guidance that can be adapted to the practice of this invention in its various embodiments and equivalents thereof.

1. A method for treating severe hypertriglyceridemia (SHTG), comprising administering to a subject in need thereof a therapeutically effective amount of a pharmaceutical composition comprising Berberine Ursodeoxycholate (BUDCA).

2. A method for treating severe hypertriglyceridemia (SHTG) and diabetes, comprising administering to a subject in need thereof a therapeutically effective amount of a pharmaceutical composition comprising Berberine Ursodeoxycholate (BUDCA).

3. The method of claim 2, wherein the diabetic disease is type 2 diabetes.

4. A method for treating severe hypertriglyceridemia (SHTG) and a fatty liver disease, comprising administering to a subject in need thereof a therapeutically effective amount of a pharmaceutical composition comprising Berberine Ursodeoxycholate (BUDCA).

5. The method of claim 4, wherein the fatty liver disease is metabolic dysfunction-associated fatty liver disease (MAFLD) or non-alcoholic fatty liver disease (NAFLD).

6. The method of claim 4, wherein the fatty liver disease is nonalcoholic steatohepatitis (NASH).

4. The method of claim 1, wherein the subject is administered from about 500 mg/day to about 2,500 mg/day of BUDCA.

5. The method of claim 4, wherein the subject is administered from about 750 mg to about 1,250 mg BID of BUDCA.

6. The method of claim 1, wherein BUDCA is in the form of Form A.

7. The method of claim 1, wherein BUDCA is a hydrate.

8. The method of claim 7, wherein BUDCA is a hemi-nonahydrate.

9. The method of claim 1, wherein BUDCA is administered as a monotherapy.

10. The method of claim 1, wherein BUDCA is administered one or more other therapy/therapies in addition to BUDCA.

11. The method of claim 10, wherein the other therapy/therapies is one or more of metformin, insulin and a GLP-1 agonist.

12. The method of claim 11, wherein the other therapy is a GLP-1 agonist.

13. The method of claim 1, wherein BUDCA is administered as an adjuvant.

14. The method of claim 1, wherein the subject is unable to take or tolerate statin.

15. A unit dosage form comprising from about 750 mg to about 1,250 mg of BUDCA.

16. The unit dosage form of claim 15, wherein BUDCA is in the form of Form A.

17. The unit dosage form of claim 16, wherein BUDCA is a hydrate.

18. The unit dosage form of claim 17, wherein BUDCA is a hemi-nonahydrate.

19. The unit dosage form of claim 15, in the form of a capsule.

20. The unit dosage form of claim 15, in the form of a tablet.

21-26. (canceled)
